(19) United States
(12) Patent Application Publication
(10) Pub. No.: US 2026/0334853 A1
(43) Pub. Date: Oct. 29, 2026

(54) QUANTITATIVE EPILEPSY DIAGNOSIS FROM SCALP EEG

(71) Applicants: THE JOHNS HOPKINS UNIVERSITY, Baltimore, MD (US); University of Pittsburgh - Of the Commonwealth System of Higher Education, Pittsburgh, PA (US)

(72) Inventors: Sridevi V. SARMA, McClean, VA (US); Kristin M. GUNNARSDOTTIR, Baltimore, MD (US); Khalil HUSARI, Baltimore, MD (US); Patrick E. MYERS, Baltimore, MD (US); Adam LI, Thousand Oaks, CA (US); Jorge A. GONZALEZ-MARTINEZ, Pittsburgh, PA (US); Niravkumar BAROT, Pittsburgh, PA (US)

(21) Appl. No.: 19/114,189

(22) PCT Filed: Sep. 26, 2023
(86) PCT No.: PCT/US2023/033668

§ 371 Date: Mar. 21, 2025

Related U.S. Application Data
(60) Provisional application No. 63/410,892, filed on Sep. 28, 2022.

(51) Int. Cl. A61B 5/00; A61B 5/372

(52) U.S. Cl. CPC A61B 5/4094; A61B 5/372; A61B 5/7267

(57) ABSTRACT

Machine learning techniques for determining a likelihood of epilepsy in an individual are presented. The techniques include: obtaining an electroencephalogram (EEG) recording of the individual, where the EEG recording lacks interictal epileptiform patterns; deriving, using an electronic processor, a plurality of features from the EEG recording; applying a trained machine learning model to the plurality of features; and providing an indication of a likelihood of epilepsy in the individual.

### CROSS-REFERENCE TO RELATED APPLICATIONS

This application is the national stage entry of International Patent Application No. PCT/US2023/033668, filed on Sep. 26, 2023, and published as WO 2024/072773 A1 on Apr. 4, 2024, which claims the benefit of U.S. Provisional Patent Application No. 63/410,892, filed on Sep. 28, 2022, which are hereby incorporated by reference in their entireties.

### GOVERNMENT SUPPORT

This invention was made with Government support under grant no. 2112011, awarded by the National Science Foundation, and under grant no. NS132228, awarded by the National Institutes of Health. The Government has certain rights in the invention.

### FIELD

This disclosure relates generally to machine learning based epilepsy evaluation.

### BACKGROUND

Epilepsy is a brain disorder characterized by a predisposition to experiencing recurrent seizures, affecting approximately 60 million people worldwide. Although 8-10% of the population will experience a seizure during their lifetime, only 2-3% of the individuals develop epilepsy. Evaluation of patients experiencing suspected seizures includes a thorough clinical history and is usually accompanied by a routine scalp EEG and brain imaging. After determining whether the episode was an epileptic seizure, the clinician evaluates the risk of subsequent seizures and the need to start anti-seizure medications.

Scalp EEG plays a central role in diagnosing epilepsy and evaluating the risk of subsequent seizures. Visual analysis and interpretation remain the gold standard in analyzing EEGs. Neurologists look for EEG abnormalities, including certain spikes and sharp waves, formally referred to as Interictal Epileptiform Discharges (IEDs), in addition to focal slowing of EEG waveform activity, all of which are known indicators of epileptic tendency. Unfortunately, the sensitivity of scalp EEG in diagnosing epilepsy varies from 29-55%, largely due to the sporadic nature of IEDs. Repeat scalp EEGs may increase the sensitivity up to 92%, but lead to significant use of financial and logistical resources for both patients and the healthcare system. Additionally, misinterpretation of the EEG as being abnormal and overinterpretation of EEG are major contributors to misdiagnosis and may result in unnecessary pharmacotherapy and reduce patient quality of life.

Furthermore, visual interpretation of EEG recordings, e.g., assessment for the presence of IEDs, is subjective and prone to variability across different EEG readers. As a result, both false positive and false negative diagnoses commonly occur, and overall misdiagnosis rates of epilepsy are nearly 30%.

Computational EEG analysis tools have emerged recently to assist clinicians in EEG analysis. The majority of the proposed algorithms automate detection of visibly apparent EEG abnormalities such as IEDs. For example, a widely used commercially available software is Persyst's Spike Detector, which identifies possible spikes in the EEG recording.

### SUMMARY

According to various embodiments, a machine learning method of determining a likelihood of epilepsy in an individual is presented. The method includes: obtaining an electroencephalogram (EEG) recording of the individual, where the EEG recording lacks interictal epileptiform patterns; deriving, using an electronic processor, a plurality of features from the EEG recording; applying a trained machine learning model to the plurality of features; and providing an indication of a likelihood of epilepsy in the individual.

Various optional features in the above method embodiments include the following. The method may include treating the individual for epilepsy based on a positive indication of the likelihood of epilepsy in the individual. The EEG recording may lack any epilepsy-indicative abnormality. The EEG recording may lack interictal epileptiform discharges (IED). The trained machine learning model may include a trained logistic regression model. The indication of the likelihood of epilepsy may include one of: epilepsy likely, or epilepsy unlikely. The method may further include removing artifacts from the EEG recording. The deriving the plurality of features may include modeling EEG data from the EEG recording as a dynamical network model. The modeling EEG data from the EEG recording as a dynamical network model may include determining a sequence of linear time-invariant dynamical network models. The deriving the plurality of features may include deriving at least one of a neural fragility metric or a source-sink index. The deriving the plurality of features may include deriving a spectral-based metric for at least one of: delta frequency band, theta frequency band, alpha frequency band, or beta frequency band. The plurality of features may include: standard deviation of fragility, standard deviation of sink-connectivity, quantile of beta, mean of frontal delta, standard deviation of frontal alpha, quantile of beta, mean of frontal sink-index, standard deviation of frontal source-influence, standard deviation of source-influence, and standard deviation of occipital source-influence.

According to various embodiments, a machine learning system for determining a likelihood of epilepsy in an individual is presented. The system includes an electronic processor and a non-transitory computer readable medium including instructions that, when executed by the electronic processor, configure the electronic processor to perform actions including: obtaining an electroencephalogram (EEG) recording of the individual, where the EEG recording lacks interictal epileptiform patterns; deriving, using an electronic processor, a plurality of features from the EEG recording; applying a trained machine learning model to the plurality of features; and providing an indication of a likelihood of epilepsy in the individual.

Various optional features of the above system embodiments include the following. The EEG recording may lack any epilepsy-indicative abnormality. The EEG recording may lack interictal epileptiform discharges (IED). The trained machine learning model may include a trained logistic regression model. The indication of the likelihood of epilepsy may include one of: epilepsy likely, or epilepsy unlikely. The actions may include removing artifacts from the EEG recording. The deriving the plurality of features may include modeling EEG data from the EEG recording as a dynamical network model. The modeling EEG data from the EEG recording as a dynamical network model may include determining a sequence of linear time-invariant dynamical network models. The deriving the plurality of features may include deriving at least one of a neural fragility metric or a source-sink index. The deriving the plurality of features may include deriving a spectral-based metric for at least one of: delta frequency band, theta frequency band, alpha frequency band, or beta frequency band. The plurality of features may include: standard deviation of fragility, standard deviation of sink-connectivity, quantile of beta, mean of frontal delta, standard deviation of frontal alpha, quantile of beta, mean of frontal sink-index, standard deviation of frontal source-influence, standard deviation of source-influence, and standard deviation of occipital source-influence.

According to various embodiments, a non-transitory computer readable medium including instructions that, when executed by an electronic processor, configure the electronic processor to perform actions for determining a likelihood of epilepsy in an individual, is presented. The actions include: obtaining an electroencephalogram (EEG) recording of the individual, where the EEG recording lacks interictal epileptiform patterns; deriving, using an electronic processor, a plurality of features from the EEG recording; applying a trained machine learning model to the plurality of features; and providing an indication of a likelihood of epilepsy in the individual.

Combinations, (including multiple dependent combinations) of the above-described elements and those within the specification have been contemplated by the inventors and may be made, except where otherwise indicated or where contradictory.

### BRIEF DESCRIPTION OF THE DRAWINGS

Various features of the examples can be more fully appreciated, as the same become better understood with reference to the following detailed description of the examples when considered in connection with the accompanying figures, in which:

FIG. 1 is a flow diagram depicting a patient selection process for inclusion of their EEG recordings as machine learning training data for a reduction to practice and for clinical evaluation as used to evaluate the reduction to practice;

FIG. 2 is a schematic diagram of a comparison of epilepsy predictions provided by the reduction to practice to clinical diagnoses;

FIG. 3 depicts boxplots illustrating epilepsy prediction by the reduction to practice;

FIG. 4 is a Receiver Operating Characteristic (ROC) curve for threshold adjustment of the reduction to practice;

FIG. 5 is chart illustrating feature weights of the reduction to practice;

FIG. 6 depicts boxplots depicting performance of the reduction to practice as measured against various metadata categories;

FIG. 7 depicts statistical plots illustrating the performance of the reduction to practice in evaluating various types of epilepsy; and

FIG. 8 is flowchart of a method for determining a likelihood of epilepsy in an individual, according to various embodiments.

### DESCRIPTION OF THE EXAMPLES

Reference will now be made in detail to example implementations, illustrated in the accompanying drawings. Wherever convenient, the same reference numbers will be used throughout the drawings to refer to the same or like parts. In the following description, reference is made to the accompanying drawings that form a part thereof, and in which is shown by way of illustration specific exemplary examples in which the invention may be practiced. These examples are described in sufficient detail to enable those skilled in the art to practice the invention and it is to be understood that other examples may be utilized and that changes may be made without departing from the scope of the invention. The following description is, therefore, merely exemplary.

Some embodiments can determine a likelihood of epilepsy in an individual based on a scalp electroencephalogram (EEG) recording of the individual. More particularly, some embodiments can noninvasively determine a likelihood of epilepsy in an individual based on an EEG, without requiring that the EEG exhibit any epilepsy-indicative abnormalities, such interictal epileptiform patterns, e.g., temporal intermittent rhythmic delta activity (TIRDA) or interictal epileptiform discharges (IED). Thus, some embodiments can determine a likelihood of epilepsy from an EEG recording independent of a presence of epilepsy-indicative abnormalities such as interictal epileptiform patterns (e.g., IED or TIRDA). Based on the determination, the patient can be diagnosed and treated, e.g., with an anticonvulsant.

Some embodiments utilize both network-based and spectral metrics, captured using non-invasive techniques, to evaluate a likelihood of epilepsy. The network-based metrics, namely fragility and source-sink, were originally developed to localize an epileptogenic zone using invasive ictal and interictal intracranial EEG recordings, respectively. Some embodiments apply such network-based metrics to non-invasive scalp EEG recordings to differentiate the networks of epileptic and non-epileptic EEGs. In particular, some embodiments derive patient-specific dynamical network models from an EEG recording and analyze the network properties to detect whether pathological patterns, inherent to an epileptic brain, are present. In general, some embodiments evaluate EEG recordings based on features derived from such network-based metrics, as well as features derived from spectral metrics, to determine whether a normal-appearing EEG recording, e.g., which lacks a presence of interictal epileptiform patterns, is indicative of epilepsy.

Some embodiments have notable advantages over prior art techniques. For example, prior art techniques that utilize neural networks do not provide the interpretability of feature relevance, as is provided by the logistic regression model of a reduction to practice described herein. As another example, existing techniques that utilize neural networks are not as accurate as the reduction to practice. As yet another example, prior art models that include healthy individuals in their control group do not differentiate conditions that mimic epilepsy (e.g., functional seizures) from epilepsy, in contrast to the reduction to practice, for example, which included data from individuals with epilepsy-mimicking conditions in the machine learning training data set. Accordingly, some embodiments provide better applicability to the real-world clinical scenarios.

These and other features and advantages are presented herein in reference to the figures.

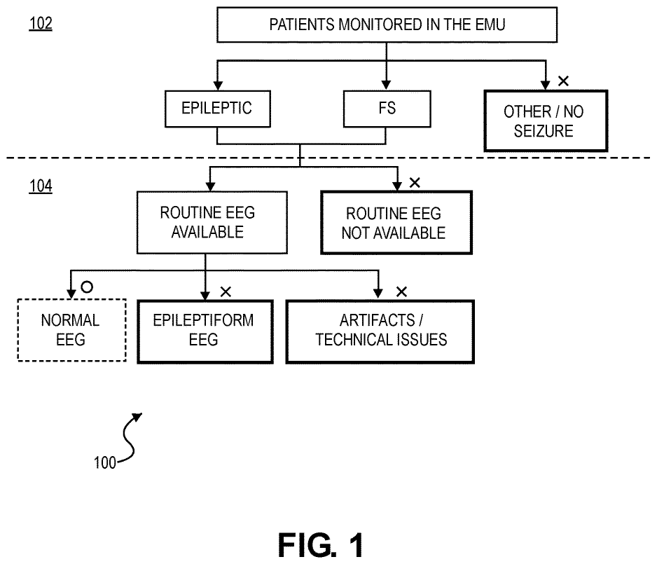

FIG. 1 is a flow diagram 100 depicting a patient selection process for inclusion of their EEG recordings as machine learning training data for the reduction to practice and for clinical evaluation as used to evaluate the reduction to practice. Throughout this disclosure, a reduction to practice is described by way of a non-limiting illustration an example embodiment. FIG. 1 illustrates the process used to select patients, where their EEG recordings were used to train and validate the reduction to practice, and where they were evaluated clinically in order to determine the prediction accuracy of the reduction to practice.

Adults patients admitted to the Epilepsy Monitoring Unit (EMU) among four clinical centers were screened as shown in FIG. 1. Patients who did not have a habitual event/seizure in the EMU and patients with both epileptic and non-epileptic seizures were excluded. Potential candidates were labeled as having either epileptic seizures or non-epileptic events, based on video EEG results in the EMU. Most patients with non-epileptic events had functional seizures (FS). A total of 198 patients were selected for inclusion, including 91 clinically diagnosed with epilepsy and 107 clinically diagnosed as not having epilepsy.

The first EEG available at the respective center was collected. Further exclusion criteria included patients without routine EEG available in the center, patients with IED or temporal intermittent rhythmic delta activity (TIRDA) based on the EEG report, and patients with EEG containing continuous, large myogenic artifacts or technical problems. In the course of standard clinical practice, EEGs were reviewed and interpreted by a fellowship trained credentialed neurologist.

In FIG. 1, boxes marked with the symbol “X” represent exclusion points. The box marked with the symbol “O” denotes the final patient population used for the reduction to practice. Initially, EMU observations were used to select patient candidates with confirmed diagnoses. Only patients who had a seizure or habitual event were included. Of the candidate EMU patients, only patients who had a prior routine scalp EEG available from the center were included. The remaining candidates were filtered by the contents of that initial routine EEG. Patients whose first routine EEG either contained specific epileptiform activity or whose EEG was deemed unusable due to continuous artifact or technical issues were excluded. The remaining patients, all of whom had a normal-presenting EEG, were included for analysis.

All participating centers record scalp EEG using a standard 10-20 montage scheme. Recording sampling rate varied based on the location: 200 Hz at one center, 256 Hz at two centers, and either 500 or 1000 Hz at the remaining center. All EEG records were downsampled to 200 Hz for analysis. Signals were referenced against an average of the C3 and C4 electrodes, and thus the reference electrodes were excluded from further analysis. Because not all centers recorded from the midline channels (Fz, Pz, and Cz), signals acquired from these contacts were discarded. The remaining 14 EEG channels, each corresponding to an electrode, were included for analysis. Data was stored in European Data Format (EDF) and organized using the BIDS-EEG scheme.

The EEG recordings underwent preprocessing as follows. To remove most myogenic artifacts, a second order bandpass filter between 1 and 30 Hz was applied to each record. The remaining artifacts, mostly ocular and cardiac, were removed through an automated process using Independent Component Analysis (ICA). Preprocessing was performed in Python via the package MNE30. The sub-package MNE-ICA was used to automatically calculate the independent components from the filtered signals and MNE-ICLabel was used to classify each component as either EEG signal or one of the following artifact types: eye, muscle, line noise, or other. MNE-ICLabel returns a percent likelihood for each classification. Components with less than 30% probability of containing EEG signal were removed. The remaining components were reconstructed into a cleaned EEG record that was used for the reduction to practice.

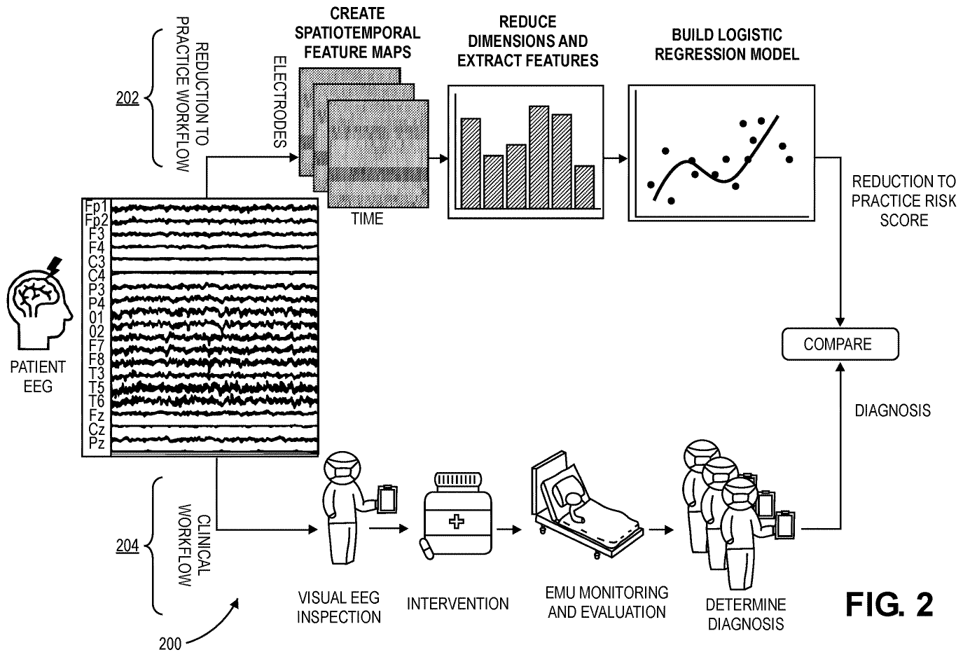

FIG. 2 is a schematic diagram 200 of a comparison of epilepsy predictions provided by the reduction to practice to clinical diagnoses. A description of both the construction workflow 202 for building the reduction to practice, and clinical workflow 204 used to evaluate the reduction to practice, follows, in reference to FIG. 2.

The reduction to practice utilized features derived from two network-based metrics, namely, a neural fragility metric and a source-sink index metric. These metrics have been shown to be useful in localizing the epileptogenic zone (EZ) in invasive ictal and interictal stereo EEG (sEEG) recordings, respectively.

Neural fragility is a concept related to the underlying dynamics of epileptic networks and the emergence of seizures. Specifically, it suggests that the onset of focal seizures may be related to the presence of a few fragile nodes, which render the epileptic network unstable and susceptible to seizure activity. During interictal periods, the network is in a “balanced” state, meaning that activity hovers around a baseline value and can respond transiently to perturbations but returns to the baseline value. However, during a pre-ictal period and during a seizure event, the network becomes “unbalanced,” with activity growing in amplitude, oscillating, and spreading throughout the brain. The notion of balance refers to the level of inhibitory and excitatory neuronal populations across the brain network.

Previously, fragility was studied using implanted electrodes, which provided intracranial EEG (iEEG) recordings. See Li A, Huynh C, Fitzgerald Z, et al. Neural fragility as an EEG marker of the seizure onset zone. Nat Neurosci. 2021; 24(10):1465-1474. doi:10.1038/s41593-021-00901-w; Li A, Gunnarsdottir K M, Inati S, et al. Linear Time-Varying Model Characterizes Invasive EEG Signals Generated from Complex Epileptic Networks. Conf Proc Annu Int Conf IEEE Eng Med Biol Soc IEEE Eng Med Biol Soc Annu Conf. 2017; 2017:2802-2805. doi:10.1109/EMBC.2017.8037439; and Sritharan D, Sarma SV. Fragility in Dynamic Networks: Application to Neural Networks in the Epileptic Cortex. Neural Comput. 2014; 26(10):2294-2327. doi:10.1162/NECO_a_00644. The fragility of each iEEG channel was computed by first estimating a linear time varying dynamical network model from iEEG data before, during and after a seizure event. The overall dynamical network model included a sequence of linear time invariant models of the form:

x(t+1)=Aᵢx(t) (1)

In Equation (1), i=1, 2, . . . , m, where there are m time windows, and Aᵢ describes how each channel influences each other dynamically within a particular time window of the iEEG. Each time window was 500 msec, by way of non-limiting example. Thus, for n channels and m time windows, the model included n×n matrices Aᵢ for i=1, 2, . . . , m. Then, from the Aᵢ matrices, an optimization routine was performed to find which channels would cause imbalance with minimal perturbations to their connections to other nodes in the network. This sequence of matrices (Aᵢ)_{i≤m} was used to calculate the fragility metric in the reduction to practice, based on external EEG recordings rather than iEEG recordings. In particular, for each Aᵢ for i=1, 2, . . . , m, a fragility was computed for each node. This gave m fragility values for each node, which were organized into an n×m matrix, where the rows represented the n nodes, the columns represented the m time windows, and the cells contained the respective fragility values.

The other network-based metric used in the reduction to practice, source-sink index, was also derived from the linear time varying dynamical network model (Aᵢ)_{i≤m}, estimated solely from interictal data. The term “source” refers to a group of brain regions that are actively influencing the electrical activity of other regions, while “sink” refers to a group of regions that are mostly being influenced by others' activity.

In the context of EEG or iEEG data, source-sink connectivity considers how electrical activity propagates through the brain network, from the sources to the sinks. It is hypothesized that the epileptogenic zone in a patient is inhibited by other regions during non-clinical seizure periods and thus are sinks. See Gunnarsdottir K M, Li A, Smith R J, et al. Source-sink connectivity: A novel interictal EEG marker for seizure localization. Published online Nov. 19, 2021:2021.10.15.464594. doi:10.1101/2021.10.15.464594. Gunnarsdottir et al. investigated this by creating an algorithm that identified two groups of nodes within an interictal iEEG network. These groups were nodes that continuously inhibit neighboring nodes (sources) and the nodes that are inhibited (sinks). Gunnarsdottir et al. estimated patient-specific dynamical network models from several minutes of interictal iEEG data, and the resulting connectivity properties as gleaned from the A matrices helped identify the top sources and sinks within the network. Specifically, Gunnarsdottir et al. quantified each node using source-sink metrics derived from the A matrices.

The reduction to practice, by way of non-limiting example regarding various embodiments, used one fragility metric and three source-sink metrics (the source-sink index, sink-index, and source-influence) for every EEG recording.

In addition to the network-based metrics described herein, and by way of non-limiting example regarding various embodiments, the reduction to practice used spectral metrics from four different frequency bands of interest: Delta (1-4 Hz), Theta (4-8 Hz), Alpha (8-12 Hz), and Beta (12-30 Hz). A multitaper Fourier Transform was calculated over non-overlapping two-second windows to accommodate the slower frequency band. These metrics each resulted in an n×m matrix where n is the number of channels and m is the number of time windows.

The process described above of determining the network-based metrics is shown in FIG. 2 as creating spatiotemporal feature maps. As further shown in FIG. 2, the next part of the process of generating the final predicative model of the reduction to practice was to reduce the dimensions of the spatiotemporal feature maps and extract features, which is described in detail presently.

To reduce the dimensionality of the feature space and to remove recording duration as a variable, for the reduction to practice, each n×m metric matrix was summarized into an n×1 vector where n is the number of channels. Two techniques were employed to achieve this dimensionality reduction: (a) time-average, and (b) Principal Component Analysis (PCA). Because no seizures were represented in the EEG recordings (interictal EEG recordings), the signals were relatively stable over time. Thus, the reduction to practice computed the average across time windows for each channel. By contrast, the second technique, PCA, aimed to capture some time-varying dynamics. To maintain the meaning of channels and only reduce dimensions on the time axis, each channel's time series was projected onto each of the first two principal components, resulting in a single value per channel for each of the first two principle components. Each dimensionality reduction was separately applied to the various metrics to derive a plurality of features, from which a selection of features were used in the reduction to practice for epilepsy prediction.

In more detail, two main feature categories were derived from each of the two dimension-reduced metric vectors: lobe-based features and channel-based features. Lobe based features were calculated by aggregating channels that belonged to the same lobe and then calculating statistics (such as the mean and standard deviation) within each lobe. Channel based features were calculated by analyzing the quantiles (10%, 50%, 90%, mean, and standard deviation) of the individual channel values over time.

This two-part feature generation method was repeated for every metric (i.e., fragility, the three source-sink, and time-frequency metrics), resulting in 39 features per metric, or 312 unique features. As described presently, a subset of these 312 features was selected for incorporation into the reduction to practice to evaluate EEG recordings from new patients.

As shown in FIG. 2, the final step in the workflow 202 of building the reduction to practice was building a logistic regression model.

The reduction to practice modeled the probability of a patient having epilepsy by way of a logistic regression model that used the features described herein as covariates. Because the possible feature space was large relative to the sample size, feature selection was implemented before constructing the final model of the reduction to practice. In particular, a recursive, greedy feature elimination procedure was developed. First, features were grouped according to their generation method (dimensionality reduction method and feature category), e.g., all PCA lobe-based features or time-averaged channel-based features. Within each such group, initial logistic regression models (described below) were built from all possible combinations of metrics. Logistic regression models were used to keep the models as simple and interpretable as possible. An L1-penalty was applied on the logistic regression models to prevent overfitting from excess features. After the search over feature groups, all resulting models were ranked by their predictive performance.

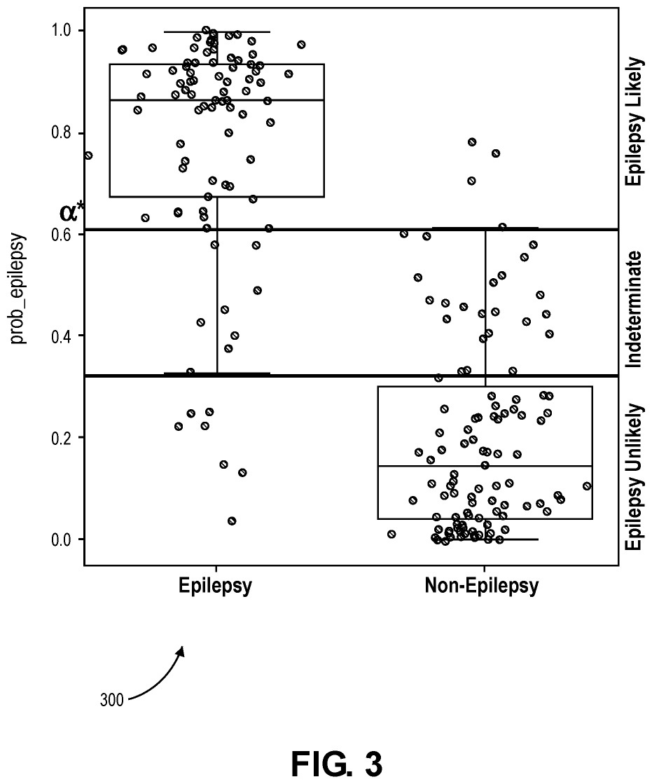

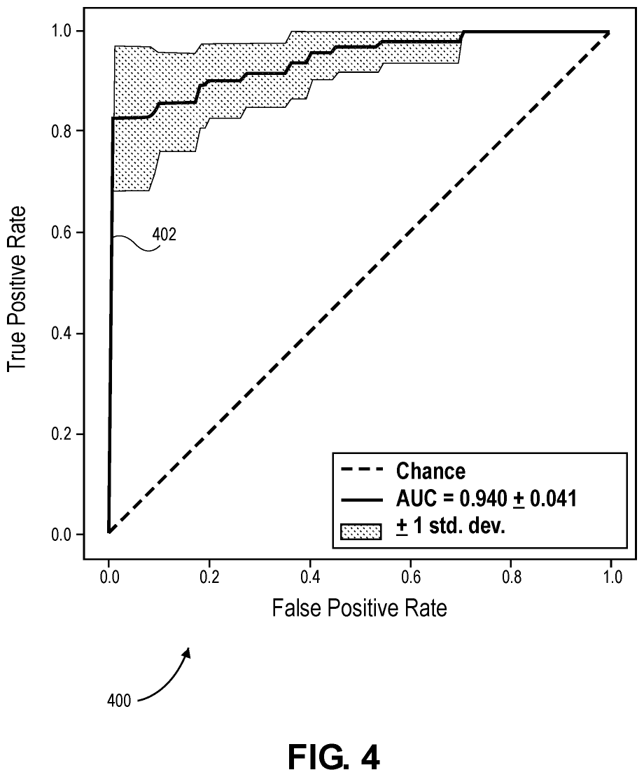

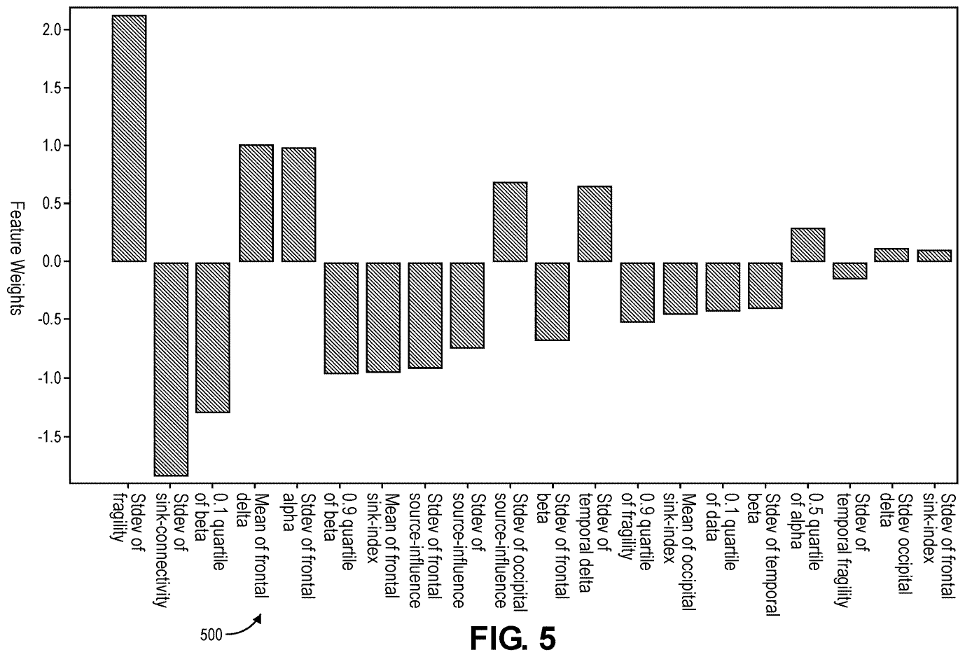

Features that consistently appeared in the top performing models, especially those with generally large and significant feature weights, were selected. This process resulted in 20 features for the final model of the reduction to practice. As shown in FIG. 2, a final logistic regression model was trained from these 20 features. The results shown and described herein in reference to FIGS. 3 and 4 regarding prediction accuracy are for this final model. The 20 features, and their weights, are shown and described herein in reference to FIG. 5 and in the Feature Table.

To summarize the model creation workflow 202, after the patient had a suspected seizure, a routine scalp EEG was collected. Network-based metrics, which are visualized as spatiotemporal heatmaps in FIG. 2, were estimated from the electrographic data. These metrics, and the spectral metrics, were condensed and summarized via multiple dimensionality reduction methods to generate features. The features were used for the final model, which can output the predicted epilepsy risk for a new patient EEG.

The clinical workflow 203 represents how the patient label was generated for each selected patient. After the EEG was collected, a qualified clinician visually inspected the record as part of their clinical determination of epilepsy likelihood. A medical intervention may have been prescribed, or the patient may have been referred directly to the EMU for further monitoring. After viewing the habitual events during this extended monitoring, the clinicians gained confidence in their epilepsy diagnoses. These diagnoses were used to compare to the output from the final model of the reduction to practice to determine its performance.

Each step of model generation workflow 202 included a ten-fold cross validation (CV) procedure using the labels from the clinical workflow 204. Each fold was randomly split into 70% training data and 30% testing data, where the training set had an even split of epilepsy and non-epilepsy patients. The model's weights were then tuned for each training set and the performance was evaluated on the corresponding test set. A varying threshold was applied to the model's output (i.e., the probability of each subject belonging to the epilepsy group), to generate evaluation metrics. Applying these thresholds classified the probabilities into a predicted diagnosis, which could be compared to the actual diagnosis (i.e., label) of each patient. The final model was evaluated using the area under the curve (AUC) of the resulting ROC curve, and the final model's accuracy, sensitivity, and specificity at the optimal threshold. For the feature selection phase, part of the model generation workflow 202, the models were ranked based on the performance on these test sets. The evaluation results are presented herein for the final model in reference to FIGS. 3 and 4.

FIG. 3 depicts boxplots 300 illustrating epilepsy prediction by the reduction to practice. Once the final model of the reduction to practice was trained, it was used to determine a probability of belonging to the epilepsy group for each patient across all ten CV folds. FIG. 3 shows these predicted probabilities of epilepsy, where each patient from the validation set is represented by a single dot. The x-axis stratifies the patients' diagnoses (based on the gold standard EMU evaluation from the clinical workflow 204 as shown and described in reference to FIG. 2). By applying a threshold to these probabilities, the prediction was converted to a classification. Probabilities above the threshold were classified as epilepsy and below the threshold were classified as non-epilepsy. With each such threshold a, the model's performance was assessed against the patients' final diagnoses by computing accuracy, sensitivity, and specificity. The threshold that resulted in the best accuracy (denoted α*) is shown in FIG. 3. With this threshold, the reduction to practice achieved an accuracy of 0.904, a sensitivity of 0.835, and a specificity of 0.963. While α* maximized prediction accuracy, a lower threshold may be used according to various embodiments if sensitivity is considered more important than specificity.

FIG. 4 is a Receiver Operating Characteristic (ROC) curve 400 for threshold adjustment of the reduction to practice. Thus, FIG. 4 provides an overview of the model's performance across a varying threshold a, represented by a receiver operating characteristic curve. The curve 402 represents the average performance of the reduction to practice across the ten-fold cross validation procedure. The gray area displays one standard deviation from this mean. The reduction to practice achieved a favorable area under the curve (AUC) of 0.940±0.041.

When used to assess epilepsy risk, the reduction to practice, as well as various embodiments, can provide great utility. By way of non-limiting example, an epilepsy highly unlikely range may be defined as any probability less than 0.32 as output by the reduction to practice. Patients within this category had a 92% ( 81/88) chance of not having epilepsy. By way of non-limiting example, an epilepsy highly likely range may be defined as any probability greater than 0.61 as output by the reduction to practice. Patients within this category had a 95% ( 76/80) chance of having epilepsy. Patients whose predicted probability fell between these ranges had a medium chance of epilepsy and may therefore be considered indeterminate, again by way of non-limiting example. If the reduction to practice were used for all patients that did not lie in the indeterminate category, then it would have achieved 93% accuracy, 92% sensitivity, and 95% specificity on 168 of the patients.

FIG. 5 is chart 500 illustrating feature weights of the reduction to practice. The final model of the reduction to practice had 20 features whose coefficients were statistically significantly different from zero. Ten of the 20 were network-based features, out of which seven were derived from source-sink metrics. Of the ten remaining spectral features, alpha, beta, and delta band features emerged as significant. The sign of each weight indicates whether the feature increases or decreases the likelihood of having epilepsy as the feature increases. For example, the coefficient for “Mean of frontal delta” is 1.01; the more the average delta band power in the frontal lobes, the probability that the patient has epilepsy modulates up by 101%. Or if the standard deviation of the fragility across EEG electrodes is large, then this modulates the probability that the patient has epilepsy up by a factor of over 200%.

The Feature Table below provides details of the 20 features used in the reduction to practice, ranked according to decreasing absolute value of their respective weights. The features are described herein in reference to FIG. 2. Each metric type is derived from one of the network-based or spectral metrics, as described in detail herein in reference to FIG. 2. The dimensionality reduction is time average, weighted time, or average, any of which can be projected onto the first or second PCA component, as described in detail herein in reference to FIG. 2. The feature type is one of the lobes or channel feature categories, as described in detail herein in reference to FIG. 2.

Feature Table

| | | | Dimensionality | Feature |
|---|---|---|---|---|
| Feature Name | Weight | Metric Type | Reduction | Category |
| Stdev of fragility | 2.13 | Fragility | Time-average | Channel |
| | | | | (quartile) |
| Stdev of sink- | −1.85 | Sink-connectivity | weighted time | Channel |
| connectivity | | | average (PCA- | (quartile) |
| | | | 1st component) | |
| 0.1 quartile of beta | −1.29 | Spectral analysis - | weighted time | Channel |
| | | Beta frequency | average (PCA- | (quartile) |
| | | | 2nd component) | |
| Mean of frontal delta | 1.01 | Spectral analysis - | weighted time | Lobes |
| | | Delta frequency | average (PCA- | (frontal) |
| | | | 1st component) | |
| Stdev of frontal alpha | 0.98 | Spectral analysis - | Time-average | Lobes |
| | | Alpha frequency | | (frontal) |
| 0.9 quartile of beta | −0.96 | Spectral analysis - | weighted time | Channel |
| | | Beta frequency | average (PCA- | (quartile) |
| | | | 2nd component) | |
| Mean of frontal sink- | −0.94 | Sink-index | weighted time | Lobes |
| index | | | average (PCA- | (frontal) |
| | | | 1st component) | |
| Stdev of frontal | −0.91 | Source-influence | Time-average | Lobes |
| source-influence | | | | (frontal) |
| Stdev of source- | −0.74 | Source-influence | Time-average | Channel |
| influence | | | | (quartile) |
| Stdev of occipital | 0.69 | Source-influence | weighted time | Lobes |
| source-influence | | | average (PCA- | (occipital) |
| | | | 1st component) | |
| Stdev of frontal beta | −0.67 | Spectral analysis - | weighted time | Lobes |
| | | Beta frequency | average (PCA- | (frontal) |
| | | | 2nd component) | |
| Stdev of temporal | 0.65 | Spectral analysis - | Time-average | Lobes |
| delta | | Delta frequency | | (temporal) |
| 0.9 quartile of fragility | −0.51 | Fragility | weighted time | Channel |
| | | | average (PCA- | (quartile) |
| | | | 2nd component) | |
| Mean of occipital sink- | −0.44 | Sink-index | weighted time | Lobes |
| index | | | average (PCA- | (occipital) |
| | | | 1st component) | |
| 0.1 quartile of delta | −0.41 | Spectral analysis - | weighted time | Channel |
| | | Delta frequency | average (PCA- | (quartile) |
| | | | 1st component) | |
| Stdev of temporal | −0.40 | Spectral analysis - | Time-average | Lobes |
| beta | | Beta frequency | | (temporal) |
| 0.5 quartile of alpha | 0.29 | Spectral analysis - | weighted time | Channel |
| | | Alpha frequency | average (PCA- | (quartile) |
| | | | 1st component) | |
| Stdev of temporal | −0.15 | Fragility | Time-average | Lobes |
| fragility | | | | (temporal) |
| Stdev of occipital | 0.12 | Spectral analysis - | weighted time | Lobes |
| delta | | Delta frequency | average (PCA- | (occipital) |
| | | | 1st component) | |
| Stdev of frontal sink- | 0.10 | Sink-index | weighted time | Lobes |
| index | | | average (PCA- | (frontal) |
| | | | 1st component) | |

Note that various embodiments may use any number of these features. For example, a model that used the top five features had a 77% accuracy; a model that used the top ten features had an 85% accuracy; a model that used the top fifteen features had an 87% accuracy; and the reduction to practice, which used all 20 features, had a 90% accuracy. Any combination of features may be used. Further additional features, not listed here, may be incorporated.

As indicated in the Feature Table, the top two features relate to standard deviation of fragility and standard deviation of sink-connectivity. In particular, a greater dissimilarity in fragility between the left and right brain hemispheres was correlated with a higher likelihood of epilepsy in patients. Further, a greater standard deviation in sink connectivity between the two brain hemispheres was correlated with a lower likelihood of epilepsy in patients.

As further indicated in the Feature Table, alpha, delta, and beta bands features emerged as statistically significant. Specifically, the higher the delta power in the frontal lobe, the higher the likelihood of epilepsy. The higher the standard deviation of alpha power between left and right frontal lobes, the higher the likelihood of epilepsy. Further, the higher overall beta power in the EEG snapshot, the lower the likelihood of epilepsy.

It is important to note that it is the collective several metrics that contributed to the accuracy of the final model of the reduction to practice, rather than any single analysis. Moreover, all of the EEGs were read as qualitatively normal.

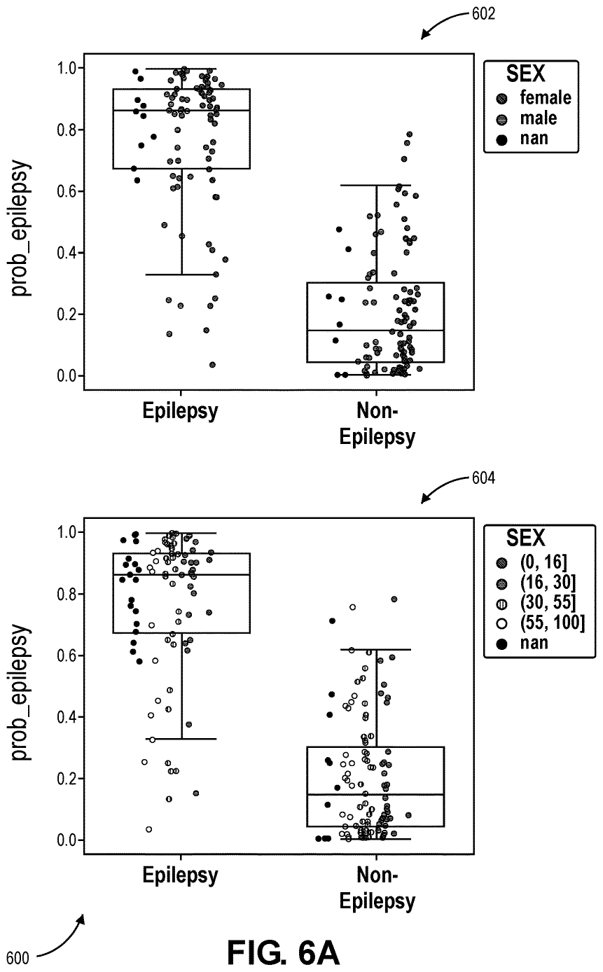

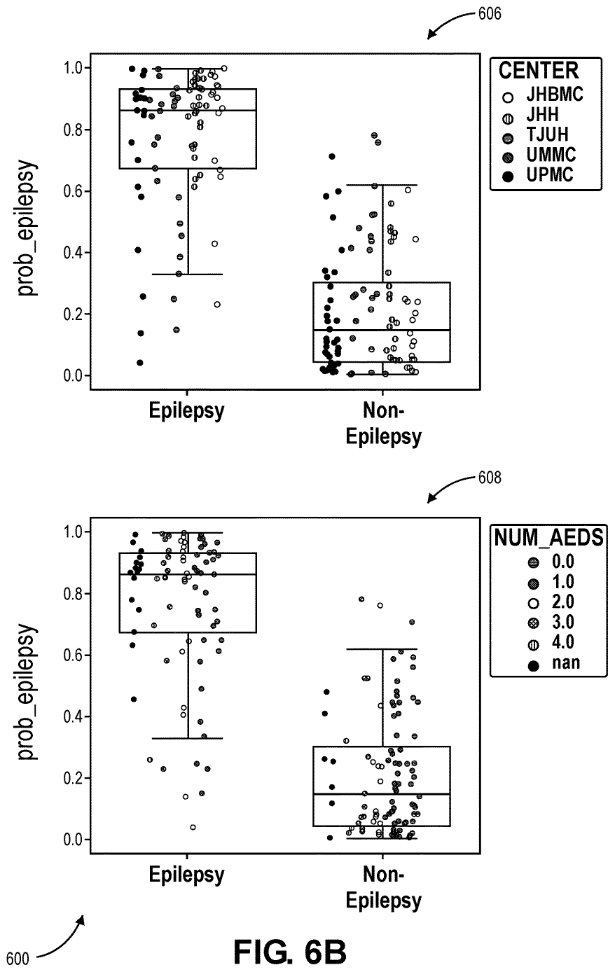

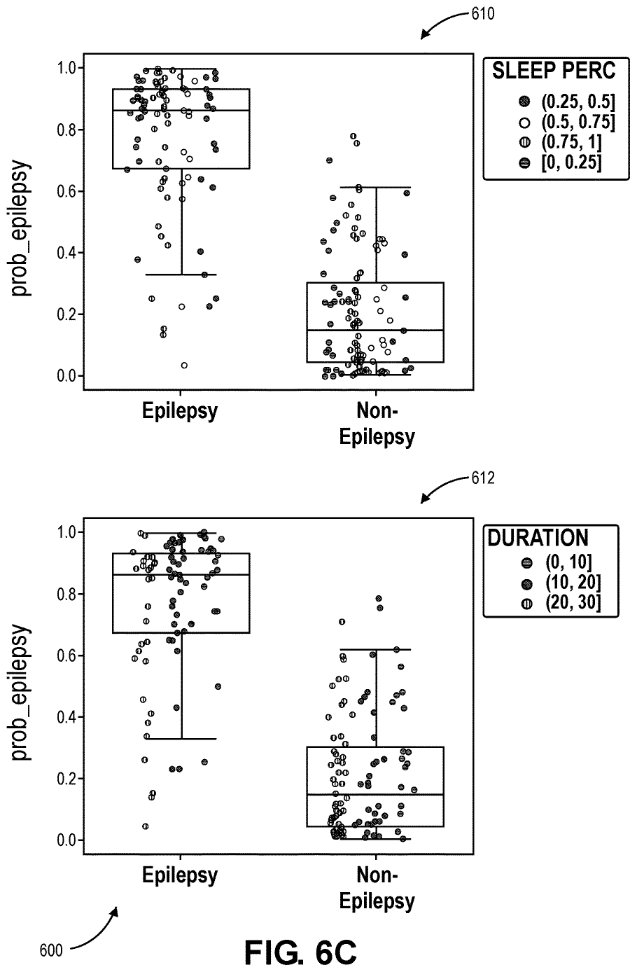

FIG. 6 depicts boxplots 600 depicting performance of the reduction to practice as measured against various metadata categories. The boxplots 600 show relative performance by sex 602, age 604 (binned into groups, center where data was collected 606, number of anti-seizure medications 608 the patient was taking at the time of the recording, percent of the recording where the patient was asleep 610 (binned into groups), and duration of the recording by minutes 612 (grouped into bins). These results show that the reduction to practice was invariant to patient type, age, sex, use of AEDs, sleep state, and duration of EEG recordings. Accordingly, these results demonstrate that the reduction to practice was generalizable among different patients.

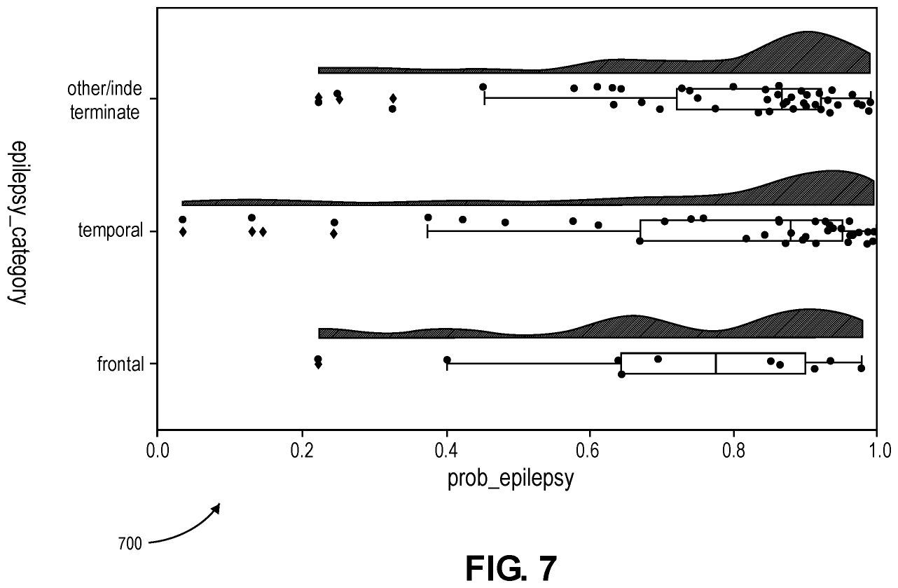

FIG. 7 depicts statistical plots 700 illustrating the performance of the reduction to practice in evaluating various types of epilepsy. In particular, the plots 700 illustrate the performance of the reduction to practice on epilepsy patients, stratified by the epilepsy type. Only temporal (N=37) and frontal (N=10) lobe epilepsy had a sufficient sample size to assess on their own. The other/indeterminate category (N=44) was composed of other focal lobes, multi-focal epilepsies, generalized epilepsy, and patients whose type of epilepsy was never determined. The reduction to practice did not perform significantly better on any particular type of epilepsy in comparison to any other type. Accordingly, these data demonstrate that the reduction to practice was insensitive to epilepsy type. That is, the reduction to practice was applicable to identifying any type epilepsy.

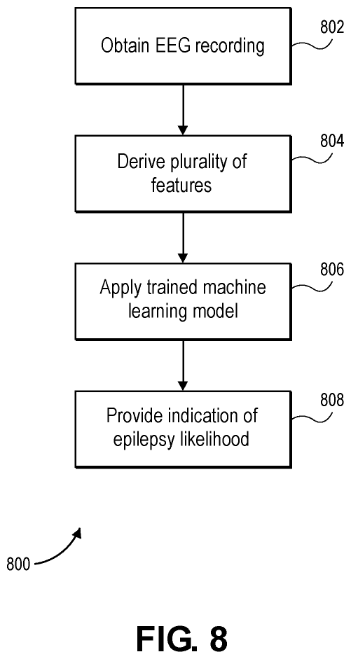

FIG. 8 is flowchart of a machine learning method 800 for determining a likelihood of epilepsy in an individual, according to various embodiments. The method may be implemented using a trained machine learning model as shown and described herein in reference to FIG. 2, for example.

At 802, the method 800 includes obtaining an electroencephalogram (EEG) recording of the individual. The recording may be obtained by retrieval from electronic storage, e.g., on a non-transitive computer readable medium. Alternately, the recording may be obtained directly from an EEG machine. According to some embodiments, the method 800 may provide an accurate assessment of whether epilepsy is present, even if the EEG recording lacks interictal epileptiform patterns, such as IED and/or TIRDA. Note, however, that the method 800 may provide an accurate assessment of whether epilepsy is present if the EEG recording does include interictal epileptiform patterns such as IED and/or TIRDA. That is, the method 800 may provide an accurate assessment of whether epilepsy is present, whether or not the EEG recording includes interictal epileptiform patterns.

At 804, the method 800 includes deriving, using an electronic processor, a plurality of features from the EEG recording. The features may be derived as shown and disclosed herein in reference to FIG. 2. By way of non-limiting example, the features may include any, or any combination, of features as presented in the Feature Table herein. Any number of such features may be included according to various embodiments, e.g., the first n features listed in the Feature Table, where n is any number from two to twenty.

At 806, the method 800 includes applying a trained machine learning model to the plurality of features. According to some examples, the trained machine learning model may be a regression model, e.g., a logistic regression model, as shown and described in detail herein in reference to FIG. 2.

At 808, the method 800 includes providing an indication of a likelihood of epilepsy in the individual. According to some embodiments, the indication of a likelihood of epilepsy may be in the form of a probability, e.g., a number between zero and one or a percentage. According to some embodiments, the indication may be in the form of a category, e.g., epilepsy likely, and epilepsy unlikely. According to some embodiments, the indication may be in the form of a category, such as epilepsy highly likely, and epilepsy highly unlikely. According to some embodiments, the possible categories may include: epilepsy highly likely, epilepsy likely, epilepsy unlikely, and epilepsy highly unlikely. Any embodiment may include a category of indeterminant. According to various embodiments, the method 800 may provide the indication by display on a computer monitor, for example. According to various embodiments, the method 800 may provide the indication electronically to a clinical computer system, e.g., over a network.

As shown and described herein, some embodiments automatically provide an indication of whether epilepsy is present in a patient based on applying a trained machine learning system to an EEG recording of the patient.

Some embodiments utilize network-based metrics of scalp EEG along with spectral features together and are are able to provide diagnostic insight for epilepsy, even when no visual abnormalities are present on the EEG record. Because scalp EEG recordings have sporadic prevalence of these visually identified abnormalities, like IEDs, their usefulness is limited in clinical practice. Some embodiments may provide effective diagnostic indications in instances where scalp EEG shows normal findings. As disclosed herein, a reduction to practice utilized three risk score thresholds (epilepsy highly unlikely, epilepsy highly likely, and indeterminate) for the interpretation of a probabilistic output from a logistic regression model. With the provided thresholds, the reduction to practice provided strong evidence (i.e., 93% accuracy) to support or reject an epilepsy diagnosis in 84% (168 of 198) normal-appearing EEG recordings.

Various embodiments utilize the presence of a characteristic network abnormality that is always (even during rest) present in an epileptic brain to provide a likelihood of epilepsy from normal-appearing EEG recording. Because various embodiments diagnose epilepsy, rather than to localize an epileptogenic zone, and because of the poor spatial density of scalp EEG as compared to invasive methods, prior art methods, e.g., those that use invasive techniques to localize epileptogenic zones, would not be expected to provide suitable results. Instead, some embodiments utilize derived features from network-based metrics, e.g., fragility and source-sink, that may identify all patients with epilepsy instead of only those with a specific type or focus. Some embodiments utilize a combination of these network features along with spectral features to predict the diagnosis of patients.

Certain examples can be performed using a computer program or set of programs. The computer programs can exist in a variety of forms both active and inactive. For example, the computer programs can exist as software program(s) comprised of program instructions in source code, object code, executable code or other formats, firmware program(s), or hardware description language (HDL) files. Any of the above can be embodied on a transitory or non-transitory computer readable medium, which include storage devices and signals, in compressed or uncompressed form. Exemplary computer readable storage devices include conventional computer system RAM (random access memory), ROM (read-only memory), EPROM (erasable, programmable ROM), EEPROM (electrically erasable, programmable ROM), flash memory, and magnetic or optical disks or tapes.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented using computer readable program instructions that are executed by an electronic processor.

These computer readable program instructions may be provided to a processor of a general-purpose computer, special purpose computer, or other programmable data processing apparatus to produce a machine, such that the instructions, which execute via the electronic processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

In embodiments, the computer readable program instructions may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Python, Smalltalk, C++, or the like, and procedural programming languages, such as the C programming language or similar programming languages. The computer readable program instructions may execute entirely on a user's computer, partly on the user's computer, as a stand-alone software package, partly on the user's computer and partly on a remote computer or entirely on the remote computer or server.

As used herein, the terms “A or B” and “A and/or B” are intended to encompass A, B, or {A and B}. Further, the terms “A, B, or C” and “A, B, and/or C” are intended to encompass single items, pairs of items, or all items, that is, all of: A, B, C, {A and B}, {A and C}, {B and C}, and {A and B and C}. The term “or” as used herein means “and/or.”

As used herein, language such as “at least one of X, Y, and Z,” “at least one of X, Y, or Z,” “at least one or more of X, Y, and Z,” “at least one or more of X, Y, or Z,” “at least one or more of X, Y, and/or Z,” or “at least one of X, Y, and/or Z,” is intended to be inclusive of both a single item (e.g., just X, or just Y, or just Z) and multiple items (e.g., {X and Y}, {X and Z}, {Y and Z}, or {X, Y, and Z}). The phrase “at least one of” and similar phrases are not intended to convey a requirement that each possible item must be present, although each possible item may be present.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function] . . . ” or “step for [perform]ing [a function] . . . ”, it is intended that such elements are to be interpreted under 35 U.S.C. § 112(f). However, for any claims containing elements designated in any other manner, it is intended that such elements are not to be interpreted under 35 U.S.C. § 112(f).

While the invention has been described with reference to the exemplary examples thereof, those skilled in the art will be able to make various modifications to the described examples without departing from the true spirit and scope. The terms and descriptions used herein are set forth by way of illustration only and are not meant as limitations. In particular, although the method has been described by examples, the steps of the method can be performed in a different order than illustrated or simultaneously. Those skilled in the art will recognize that these and other variations are possible within the spirit and scope as defined in the following claims and their equivalents.

1. A machine learning method of determining a likelihood of epilepsy in an individual, the method comprising:
obtaining an electroencephalogram (EEG) recording of the individual, wherein the EEG recording lacks interictal epileptiform patterns;
deriving, using an electronic processor, a plurality of features from the EEG recording;
applying a trained machine learning model to the plurality of features; and
providing an indication of a likelihood of epilepsy in the individual.

2. The method of claim 1, further comprising treating the individual for epilepsy based on a positive indication of the likelihood of epilepsy in the individual.

3. The method of claim 1, wherein the EEG recording lacks any epilepsy-indicative abnormality.

4. The method of claim 1, wherein the EEG recording lacks interictal epileptiform discharges (IED).

5. The method of claim 1, wherein the trained machine learning model comprises a trained logistic regression model.

6. The method of claim 1, wherein the indication of the likelihood of epilepsy comprises one of: epilepsy likely, or epilepsy unlikely.

7. The method of claim 1, further comprising removing artifacts from the EEG recording.

8. The method of claim 1, wherein the deriving the plurality of features comprises modeling EEG data from the EEG recording as a dynamical network model.

9. The method of claim 8, wherein the modeling EEG data from the EEG recording as a dynamical network model comprises determining a sequence of linear time-invariant dynamical network models.

10. The method of claim 1, wherein the deriving the plurality of features comprises deriving at least one of a neural fragility metric or a source-sink index.

11. The method of claim 1, wherein the deriving the plurality of features comprises deriving a spectral-based metric for at least one of: delta frequency band, theta frequency band, alpha frequency band, or beta frequency band.

12. The method of claim 1, wherein the plurality of features comprise: standard deviation of fragility, standard deviation of sink-connectivity, quantile of beta, mean of frontal delta, standard deviation of frontal alpha, quantile of beta, mean of frontal sink-index, standard deviation of frontal source-influence, standard deviation of source-influence, and standard deviation of occipital source-influence.

13. A machine learning system for determining a likelihood of epilepsy in an individual, the system comprising an electronic processor and a non-transitory computer readable medium comprising instructions that, when executed by the electronic processor, configure the electronic processor to perform actions comprising:
obtaining an electroencephalogram (EEG) recording of the individual, wherein the EEG recording lacks interictal epileptiform patterns;
deriving, using an electronic processor, a plurality of features from the EEG recording;
applying a trained machine learning model to the plurality of features; and
providing an indication of a likelihood of epilepsy in the individual.

14. The system of claim 13, wherein the EEG recording lacks any epilepsy-indicative abnormality.

15. The system of claim 13, wherein the EEG recording lacks interictal epileptiform discharges (IED).

16. The system of claim 13, wherein the trained machine learning model comprises a trained logistic regression model.

17. The system of claim 13, wherein the indication of the likelihood of epilepsy comprises one of: epilepsy likely, or epilepsy unlikely.

18. The system of claim 13, wherein the actions further comprise removing artifacts from the EEG recording.

19. The system of claim 13, wherein the deriving the plurality of features comprises modeling EEG data from the EEG recording as a dynamical network model.

20. The system of claim 19, wherein the modeling EEG data from the EEG recording as a dynamical network model comprises determining a sequence of linear time-invariant dynamical network models.

21. The system of claim 13, wherein the deriving the plurality of features comprises deriving at least one of a neural fragility metric or a source-sink index.

22. The system of claim 13, wherein the deriving the plurality of features comprises deriving a spectral-based metric for at least one of: delta frequency band, theta frequency band, alpha frequency band, or beta frequency band.

23. The system of claim 13, wherein the plurality of features comprise: standard deviation of fragility, standard deviation of sink-connectivity, quantile of beta, mean of frontal delta, standard deviation of frontal alpha, quantile of beta, mean of frontal sink-index, standard deviation of frontal source-influence, standard deviation of source-influence, and standard deviation of occipital source-influence.

24. A non-transitory computer readable medium comprising instructions that, when executed by an electronic processor, configure the electronic processor to perform actions for determining a likelihood of epilepsy in an individual, the actions comprising:
obtaining an electroencephalogram (EEG) recording of the individual, wherein the EEG recording lacks interictal epileptiform patterns;
deriving, using an electronic processor, a plurality of features from the EEG recording;
applying a trained machine learning model to the plurality of features; and
providing an indication of a likelihood of epilepsy in the individual.
